(19) United States
(12) Patent Application Publication
(10) Pub. No.: US 2017/0337834 A1
(43) Pub. Date: Nov. 23, 2017

(54) INTERACTIVE BRAIN TRAINER

(71) Applicant: Rajaa Shindi, Las Cruces, NM (US)

(72) Inventor: Rajaa Shindi, Las Cruces, NM (US)

(21) Appl. No.: 15/597,683

(22) Filed: May 17, 2017

Related U.S. Application Data
(60) Provisional application No. 62/337,770, filed on May 17, 2016.

(51) Int. Cl. G09B 5/02; A61B 5/00; A61B 5/0478; A61B 5/0482

(52) U.S. Cl. CPC G09B 5/02; A61B 5/0482; A61B 5/0006; A61B 5/002; A61B 5/0478

(57) ABSTRACT

A brain training system and method are provided that incorporates a first phase and a second phase of brain training. The first phase is designed to increase attention by requiring the completion of an attention-based activity or task. This may require the identification of match or mismatched images. The second phase is designed to increase working memory by requiring the completion of a working memory-based task that requires the repetition of an illumination sequence. Each phase may be implemented in a self-contained handheld device, similar to the shape of a tablet, or may be implemented in a computer application, such as software. The answers of each phase are recorded in a central database and the system may provide recommendations based on the answers.

### CROSS REFERENCE TO RELATED APPLICATIONS

This application claims the benefit of prior co-pending U.S. Provisional Patent Application Ser. No. 62/337,770, filed on May 17, 2016; the disclosure of which is incorporated herein by reference.

### BACKGROUND

### Technical Field

The present disclosure relates generally to computer-based training systems. More particularly, the present disclosure provides a system and method for improving attention and working memory through the use of either an application or one or more three dimensional hand-held devices incorporated into a curriculum.

### Background Information

Working memory is a cognitive system with a limited capacity that is responsible for temporarily holding information available for processing. Working memory is important for reasoning and the guidance of decision making and behavior. Working memory is often used synonymously with short-term memory, but some theorists consider the two forms of memory distinct, assuming that working memory allows for the manipulation of stored information, whereas short-term memory only refers to the short-term storage of information. Working memory is a theoretical concept central to cognitive psychology, neuropsychology, and neuroscience.

Attention is the behavioral and cognitive process of selectively concentrating on a discrete aspect of information, whether deemed subjective or objective, while ignoring other perceivable information. It is the taking possession by the mind in clear and vivid form of one out of what seem several simultaneous objects or trains of thought. Focalization, concentration of consciousness are of its essence. Attention has also been referred to as the allocation of limited processing resources.

Recent interest in how brain functions relate to mathematics achievement has brought into focus the role of working memory and attention. In some example embodiments, systems and methods combine brain training to increase activity in parts of the brain and the rule of tangible Physical Manipulatives (PM). Tangible physical manipulatives allow children to take advantage of their real-world experience during the learning process. The disclosed systems and methods may help children as well as other subjects learn to increase working memory and focus attention independently.

Brain training applications and physical manipulative interfaces are disclosed herein. The physical manipulative interfaces are described. A brain-computer interface is embedded with the brain training system to read the child's attention state in real time or substantially real time. The disclosed applications and interfaces are evaluated with eighteen children with low mathematical achievement and attention-deficit disorder. The results may indicate significant improvements in task completion compared to traditional methods, and significantly lower overall subjective workload. Some, if not all, children and teachers report that they prefer the new way to interact with the application over at least one traditional way. The brain training system disclosed herein may be considered more flexible, noninvasive, drug-free, child-friendly way to reduce symptoms associated with attention deficit disorder.

Arithmetic is an academic skill that relies on a range of cognitive processes such as working memory and attention. High working memory capacity and attention span are required for complex mathematical tasks. Mathematical thinking affects the daily activities of young children and plays a role in many facets of adult life. Working memory (WM) is part of the human memory system that provides temporary storage for the manipulation of information to process complex tasks such as language comprehension, learning, and reasoning 6. Subjects can be trained on how to increase attention span and to improve visuospatial working memory to improve learning behaviors in the context of math classrooms.

Attention Deficit Hyperactivity Disorder (ADHD) affects 3-5% of school-age Children—approximately 2 million children in the United States 1. An aspect of the subject matter disclosed herein is to improve the brain's cognitive functions, such as working memory capacity and attention span, among ADHD-affected individuals. The increase in activity may cause the brain to form new neural pathways and therefore new learning, which may be demonstrated as improved performance on achievement tasks.

In some example embodiments, two paradigms—EEG neurofeedback brain training and the power of tangible physical manipulative (PM) interfaces—are combined to provide a physical manipulative brain training system. Tangible physical manipulative interfaces link the digital and physical worlds, positively affecting and increasing student learning outcomes and supporting neuron rehabilitation. Brainwave activities can be measured by electroencephalography (EEG) and read using a brain-computer interface (BCI), however their impact on brain training techniques has not been studied extensively.

In some example implementations, a physical manipulative brain training system may be used to positively affect students' engagement and performance, leading to an increase in working memory and attention. This effect may be expected to be greater than the corresponding effect derived from the use of non-physical manipulative interface (N-PM-Brain Training System).

A purpose of the physical manipulative and non-physical manipulative systems is to improve cognitive functions such as attention and working memory in children with low mathematics achievement, such as those diagnosed with attention disorder. The subjects may have increased attention and working memory capacity (beta brainwaves) and suppress drowsiness (theta brain waves) through training. A first aspect is to provide a child-friendly interactive physical manipulative brain training system that provides EEG-derived neurofeedback. This system may be evaluated using experiments that compare performance, task load, etc. in the physical manipulative brain training system to non-physical manipulative brain training system. Both systems can be implemented based on psychological tasks—the matching psychological task and that spatial-span psychological task. Students' attention feedback may be observed on the screen in the non-physical manipulative brain training system or through light-emitting diodes (LEDs) in the physical manipulative brain training system.

Another aspect is to increase the user's communication bandwidth and quality of the interface by evaluating the effects of physical manipulation on the user's performance and direct engagement. Direct engagement occurs when a user experiences direct interaction with the objects in a domain. Challenging physiological tasks and motivating visual feedback, such as LEDs, are embedded in the design to engage children in the brain training system. Improving learning behaviors in the classroom through data provides feedback needed teachers and students to design supportive learning environments.

The human brain has many capabilities and abilities and contains approximately 100-billion neurons that communicate through synapses. Research shows that both genetics and environmental factors have an impact on the development and physical structure of the brain. Different areas of the brain are responsible for different cognitive functions; the frontal and parietal areas are known to be involved in the control of attention and visuospatial working memory. Research shows that, during childhood, the development of the functionality in these areas may play an important role in cognitive development. Attention is “the human ability to concentrate on a certain objects and allocate processing resources accordingly.” Visuospatial working memory is the component that allows us to temporarily hold and manipulate information about places, finding our way around the environment. It allows us to observe objects and the spatial relationships among them.

One of the abilities of the brain is neuroplasticity, which is the concept behind brain training to improve cognitive functions and is defined as altering the brain's neural synapses and pathways through behavioral changes. Brain training reflects the capability of the brain to change and adapt itself, given the right challenge for a specific individual. The adaptability of the brain provides the systematic practice necessary for establishing new neural Circuits and for strengthening the synaptic connections among the neurons in the circuit. Studies indicate that the greatest changes occur through repeated practice of a skill over an extended period of time. Imaging and electrophysiological studies following cognitive training indicate that physical changes in the brain are associated with increases in the studied cognitive abilities.

In some example embodiments, neurofeedback can be implemented (for example, an electroencephalography (EEG) biofeedback). This neurofeedback may be used to measure electrical activity of the brain. Users learn to generate specific brainwaves through various mental strategies, while monitoring the outcome of their efforts in near real time. Different brainwaves are associated to different states of the brain.

The cognitive symptoms of attention deficit hyperactivity disorder (ADHD) are primarily attention-related difficulties and working memory capacity. The current ADHD medications have several limitations, such as short-term benefits and potential long-term risk; therefore, scientists have attempted to use alternative ways to treat ADHD. Research shows that the performance of working memory capacity for children with ADHD can be improved via training. Both neuroscience research and studies of brain training programs show that brain training at a younger age improves working memory and attention for children with ADHD; the training can be realized using psychological test tasks, software games or waves feedback on the computer screen.

Some exemplary commercially available software include BrainMaster (www.brainmaster.com) which provides neurofeedback brain training for all ages, SmartMind/Cogmed (www.cogmed.com) which provides working memory training for children, Cogmed RM/QM (www.cognfit.com) which provides cognitive training, Reading Assistant by Scientific Learning (http://www.scilearn.com/products/reading-assistant) which provides children and adult reading fluency training, and Learning RX (www.learningrx.com) which provides cognitive skills training.

Neurofeedback works through the principle of brain neuroplasticity, which has been thoroughly researched. Studies show that cognitive training using noninvasive imaging and EEG methods results in physical changes in the brain associated with increases in cognitive ability. In a study by Olesen, Westberg, and Klingberg, noninvasive methods, such as functional magnetic resonance imaging (fMRI), measured changes in children's brain activity. Several weeks of daily practice increased Working Memory (WM)-related activity in the frontal cortex.

Working memory consists of several components, including spatial storage for verbal and visuospatial resources and an attentional section, with each component playing a role in mathematical cognition. Research shows that visuospatial working memory capacity is highly correlated with mathematical reasoning abilities and can predict future development of mathematical performance. The brain neurons are grouped to accomplish the task as one thinks, learns, or remembers. If the task is difficult or unfamiliar, nearby neurons are drawn into the process to assist. Many learning activities in which children engage, such as reading, mathematics, science, place considerable loads on working memory. In mathematics, many activities require a child to hold information in mind while processing a task. These are the kinds of activities where children with poor working memory struggle most, and they often fail to complete tasks properly because they have lost crucial information needed to guide their actions from working memory. Often, it appears that the child has not paid attention when, in fact, he or she has simply forgotten what is to be done.

Many researchers have investigated the activity of EEG brainwaves. EEG involves a set of signals that may be classified according to frequency. Frequency ranges are referred to as delta (Δ), theta (θ), alpha (α), and beta (β), from low to high frequencies, respectively. Some details of these waves are described below.

The lowest delta waves lie below 4 Hz. They occur in adults during deep sleep; in babies, they decrease with age. A large amount of delta activity in conscious adults is abnormal and is related to neurotic diseases. Theta waves have frequencies from 4 to 7 Hz. A large amount of theta frequencies can be seen in young children, but this frequency can also occur during emotional stress in some adults. Theta waves are even seen during times of drowsiness, daydreaming, or light sleep and have been associated with a wide range of cognitive processes, such as mental calculation. Alpha waves occur at a frequency range of 8 to 13 Hz and are present when a person is in a relaxed state and not actively thinking. Beta waves, with very low amplitude and high frequency, range from 13 to 30 Hz and are recorded in the frontal and central regions of the brain. They occur when a person is interacting with the environment and concentrating, thinking, or solving problems.

In EEG neurofeedback training programs, the participant wears an EEG net and views computer feedback of his or her brain activity. The participant is encouraged to learn to “control” this readout by receiving coaching from a clinician on maintaining effort and focus using metacognitive strategies. Users learn to generate specific brainwaves through various mental strategies while monitoring the outcome of their efforts in near real time.

EEG has been used in clinical and research settings since the 1960s because of the rich information obtained regarding how people perform tasks. Recent research has reported that EEG neurofeedback training that focused on inhibiting theta waves and increasing beta waves is an effective treatment for children with attention deficit disorder. Studies comparing neurofeedback to medication have provided evidence for the potential of such training as a non-pharmacological treatment for a variety of neurotic disorders.

In some example embodiments, computer-based educational approaches can be used to support an increase in beta(attention)brainwaves, and decrease the theta (drowsiness) brainwaves. Research indicates that children who received EEG neurofeedback training to prevent theta waves (4-8 Hz) and increase beta waves (13-30 Hz) show significant improvement in school performance, grades, and achievement test scores

Cognitive psychology is a major component in human computer interaction research, as it provides and applies psychological ideologies and principles to develop systems that describe and predict human performance.

Attention and memory have helped human computer interaction researchers to develop approaches about what can and should be presented in an interface. Comprehensive research has been conducted in areas of focused/divided attention. In the context of human information processing, attention is the process that, at a given moment, enhances some information and inhibits other information. The enhancement enables one to select some information for further processing, while the inhibition enables one to set some information aside. Although what it means to “pay attention” to an objector event may be clear, the study of attention has a long and complex history in cognitive psychology, filled with debate and disagreement.

A brain computer interface (BCI) refers to human and computer interaction. Passive brain—computer interfaces consider brain activity as an additional source of information, to enhance and adapt an interface instead of controlling, providing direct neural monitoring. Human computer interaction research shows that combining human computer interaction/human computer interaction can lead to the development of more efficient and intelligent human computer interaction systems by using a variety of sources and modalities—for example, electroencephalogram (EEG), functional magnetic resonance imaging or functional MRI (fMRI), electromyography (EMG), electrooculography (EOG), and visual audio-touch sensors. EEG is most commonly used for brain measuring or monitoring in human computer interaction. A recent major review of brain computer interface predicts that in the near future, BCI systems may therefore become a new mode of Human-Machine interaction with levels of everyday use that are similar to other current interfaces. A recent study concluded that the BCI based attention training game is a potential new treatment of ADHD children.

NeuroSky Brain Computer Interface (NSBCI) is a non-invasive, dry, biosensor used to read electrical activity in the brain to determine states of attention. NSBCI is able to measure and record raw EEG brainwaves by using three dry electrodes located on the left ear and forehead. Using NSBCI's original algorithm, attention is calculated based on EEG brainwaves. Every second, the headset computes the attention measures based on the user's brain activity. The output is a number from 0-100. This NSBCI measurement is utilized by several researchers to measure usability and accuracy.

### SUMMARY

In some example embodiments, there is provided an interactive system, method, and article of manufacture for training a brain. The interactive system, method, and article of manufacture may include instructions encoded on a non-transitory computer readable storage medium, that when executed by one or more processors is configured to assist children increase their brain activity through an attention-based task/activity (a first phase or phase one). The attention based activity requires the test subject to identify a matching image. This is utilized as a feature to match psychological tasks and requires the user to “concentrate” or “focus attention” on complex images. A region in the front of the brain, known as the mid-ventrolateral frontal cortex, responds when the image that the subject is looking for appears, even though this image changes often throughout the course of the experiment. To assist with measuring brain activity a brain computer interface may be integrated with the system implementation to measure the subject's attention state. This attention based activity/task may be implemented solely in an application based technology (i.e., computer, display, and keyboard) or may be implemented in a hardware based technology (i.e., a self-contained tablet-like device).

The interactive system, method, and article of manufacture may also include instructions encoded on a non-transitory computer readable storage medium, that when executed by one or more processors is configured to assist children increase their brain activity through an working memory-based task/activity (a second phase or phase two). The working memory-based task/activity is based on the spatial-span concept (a used tool in clinical neuropsychology that exercises visuospatial working memory). Neuroscientific studies show that visuospatial memory can be improved through “chunking” strategies—a process to contain group recoding items into memorable clusters of information challenging spatial-span step is created. To assist with measuring brain activity a BCI is integrated with the system implementation to measure the subject's attention state. This working memory-based activity/task may be implemented solely in an application based technology (i.e., computer, display, and keyboard) or may be implemented in a hardware based technology (i.e., a self-contained tablet-like device).

In accordance with one aspect, the present disclosure provides a brain training matching application that allows children to train to improve their focusing attention and a working memory application that trains/enhances of working memory through sequential repetition. The present disclosure may provide utilize a Neurosky mindset EEG equipment (or other neurofeedback headset device), featuring only one electrode on the forehead, however more electrodes are possible. EEG signals from the user may be sent to a main computer or database via Bluetooth. The signal may then processed and calculated using an algorithm. The information can be used as a real-time visual feedback for the subjects while they worked the two separate physiological tasks. The visual feedback in a physical application (such as a handheld tablet-like unit) can use LEDs that illuminate if the subject reaches a certain attention threshold. In a nonphysical application (such as a computer screen based task), the attention bar level changed, based on attention thresholds. One exemplary advantage of this embodiment, and the other embodiments as well, it that it can be effective in helping children to learn how to train the brain to focus attention and retain information in a motivated way. Empirical results reflect an improvement in task completion over traditional training methods, and a significantly lower frustration level among subjects. Additionally, the brain training matching application of the present disclosure appears to be a flexible, noninvasive, drug free, child-friendly solution to reduce symptoms associated with attention deficit disorder.

In some example embodiments, there is provided systems and methods that combine brain training to increase activity in parts of the brain and the rule of tangible Physical Manipulatives(PM).

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an enterprise resource software system or other business software solution or architecture, it should be readily understood that such features are not intended to be limiting. The claims that follow this disclosure are intended to define the scope of the protected subject matter.

In accordance with one aspect, an embodiment of the present disclosure may provide a brain training system and method that incorporates a first phase and a second phase of brain training. The first phase is designed to increase attention by requiring the completion of an attention-based activity or task. This may require the identification of match or mismatched images. The second phase is designed to increase working memory by requiring the completion of a working memory-based task that requires the repetition of an illumination sequence. Each phase may be implemented in a self-contained handheld device, similar to the shape of a tablet, or may be implemented in a computer application, such as software. The answers of each phase are recorded in a central database and the system may provide recommendations based on the answers.

In accordance with one aspect, an embodiment of the present disclosure provides a multi-phase system for training and teaching a student or test subject how to increase attention and working memory. A first phase relates to implementations for increasing attention. A second phase relates to implementation for increasing working memory. The terms first and second are sequential as used herein, such that the second phase occurs subsequent to the first phase. The first and second phases are part of an overall brain training exercise for increasing attention and working memory. While various studies have been conducted on attention exercises (i.e., phase one) and working memory exercises (i.e., phase two), the present disclosure implements these phases in manner which have been shown through empirical testing, to lead to increased attention and working memory based on the frequency of the testing and the testing periods.

In accordance with one aspect, an embodiment of the present disclosure assist test subjects (such as ADHD children) with improving their working memory by completing exercises in a structured manner. The system further effectuates increase in short-term memory of test subjects. This is accomplished by encouraging the brain to process information more effectively, and the effective processing is accomplished by completing the exercises (i.e., phase one and phase two) in the empirically established frequency and period.

The first phase is an attention-based exercised depicting two images of geometric shapes or objects. A first image has a first number of objects at specific locations. A second image has a second number of objects at specific locations. The test subject must identify whether the first number of objects equals the second number of objects at the same locations. If the first and second images are identical, then the test subject identifies the “match” and identifies the same via a “Match” button. Otherwise, if the subject identifies that the first and second images do not match (i.e., are dissimilar), then the subject identifies the mismatch via a “mismatch” button.

Furthermore, one embodiment provides that the shape of the objects as well as the size as well of the location of the object may vary between the first and second images. For example, each of the two images may have 4 objects, but the object could have different sizes relative to the two images or different locations of the objects between the two images, which would indicate a mismatch. Furthermore, utilizes shapes improves the brain's ability to “chunk” the objects, as opposed to using faces, which a test subject cannot “chunk” the information.

With respect to the second phase, a plurality of illuminated objects is presented to the test subject. In one embodiment, they are presented in a sequential manner. For example, illuminating a first button, a second button, and a third button. However, other embodiment may provide for illumination of more than one button at a time. Then, the test subject must reproduce (i.e., repeatably enter) the pattern into the working memory application or device.

The response time is measured in each the first phase and the second phase from the time at when each task is initiated and stops when the test subject records an answer. It has been determined that response time is usually lessened in a physical hardware device which resultants in the test subject having more control over the brain-computer interface. The response time is then logged in a central database, or server. The server can be remote from the device or testing station at which the subject is completing the brain training tasks.

The sequentiality or sequence of phase one occurring before phase two may be critical to a method of the present disclosure. In one example, completing the attention-based task first trains a student first how to pay attention. This helps the program/system of the present disclosure to initiate an engagement with the test subject in order to get them to pay attention. Thereafter, the program/system of the present disclosure to initiate a second engagement with the test subject in order to get them train their working memory. However, the attention based step should occur first in order to get the brain “warmed up” so it can be ready to receive the training for the working memory tasks. It is believed that this sequential combination is an improvement in brain training technique which results in improved brain functioning for the test subject.

In one aspect, an exemplary embodiment of the present disclosure provides a method of training or exercising a brain through mental tasks/activities, the method including the steps of first performing a first task that is an attention-based task, then secondly completing a second task that is a working memory-based task. The attention based task is accomplished by matching and mismatching images that includes first and second images including blocks or shapes (in one example the first and second images do not human faces). The working-memory based task is a sequentially based illumination sequence requiring the test subject or user to repeat the illumination sequence to score a “correct” answer. During each task, a neurofeedback device provides real-time neural feedback of brain waves or brain activity to the user/test subject. This provides encouragement to the user so they can watch their progress and strive to improve their brain waves (i.e. focus) as they continue with the tasks/activity.

In one exemplary embodiment, the braining training system of the present disclosure may be incorporated or implemented in a school curriculum. In one example, the present disclosure sets aside 20 minutes per student during their existing computer lab time. Then, during the legacy computer lab course during school (for elementary school students), the attention based-task (i.e. phase one) may be practiced for at least twenty days. Then, after completing twenty 20-minutes session of phase one (the attention based task on either the application based version or the hardware based version), the student can then move on to phase two in order to complete the working memory based tasks. Then, the working memory based tasks are completed in another twenty sessions. Thus, 40 sessions total (20 attention based session and 20 working memory based sessions).

In accordance with yet another aspect, an embodiment of the present disclosure may provide a brain training device that may be applicable to both adults and children who are in need, or otherwise desire, to strengthen their attention and working memory through the use of a structured program curriculum. In this scenario, EEG neurofeedback is utilized to provide real time information to the test subject about the brainwaves experienced during the accomplishment of a task, wherein the task can either be the attention based task or the working memory based task. In one example, the EEG neurofeedback may be substituted for other types of biological feedback for other sensors configured to receive personal information detected by a sensor of the test subject in real time during performance of the activity/task.

In accordance with another aspect of the present disclosure, an embodiment may provide a portable and mobile system configured to train a test subject via a brain computer interface to increase working memory and attention while monitoring brain activity or brainwaves with a portable and mobile EEG neuro feedback device. This may provide an exemplary advantage over a fixed and rigid system such as an MRI machine, which would require the user or test subject to attend a hospital or doctor's office in order to complete the tasks. This would clearly be a disadvantage inasmuch as it has been empirically determined that the period and frequency of the tests associated with the present disclosure occur over a span of about two weeks during a number of periods of approximately twenty minutes at each session.

In accordance with an aspect of the present disclosure, a system for training a brain to improve attention and working memory includes a plurality of test subject work stations and a central administrator overseeing the simultaneous implementation of multiple activities and tasks of the test subjects. For example, the central work station may be monitored in real time by a teacher, such as a computer lab teacher in an elementary school, whereas the plurality of work stations are occupied by students acting as test subjects to improve either one of their attention and working memory through the attention based tasks and activities and the working memory based tasks and activities respectively. Stated otherwise, an exemplary embodiment of the present disclosure enables at least two people to simultaneously monitor the brainwave neurofeedback of the test subject. A first person observing brainwave neurofeedback is the test user themselves, who monitor real time brainwave feedback in an attention indicator integrated into either the program or the hardware device itself. Additionally, a second user is the administrator or teacher who monitors the real time brainwave feedback of the test subject in a control computer that is linked, either wirelessly or in a wired manner, via a network, such as the internet, to the computing device upon which the test subject is interacting to complete the attention based task or the working memory based task.

In accordance with another aspect of the present disclosure, the system and devices for a brain training system includes the first phase and the second phase wherein in each phase the test subject must accomplish certain tasks. In one exemplary embodiment, the tasks increase in difficulty as the user progresses in each round and gets answers correctly. However, if an answer is incorrect, then the difficulty may decrease (i.e., become easier). In most instances, the difficulty corresponds to attention however, it is still entirely possible for attention to remain high and beta waves within the brain still be functioning at an optimal level even though the test subject may have answered incorrectly. The attention indicator on either one of the attention based devices or applications or the working memory based devices or applications monitor in real time the brain wave activity independent from the answers selected by the test subject during the activity/task. Thus, it can be said that the brainwave feedback is independent from the answers. They are loosely coupled much as the feedback represents the student's concentration level during the performance of a task within the activity.

In accordance with another aspect of the present disclosure, an embodiment may provide a task completion time that is recorded in a central database for each student or test subject. The task completion time is associated with the time each subject takes to complete a required task. This information can then be uploaded to central server for storage thereon. In one aspect, an embodiment of the present disclosure may provide a threshold or a standard rubric upon which the completion time per test subject is compared. The central server may perform analysis to determine whether the completion time per test subject is above or below the standard threshold for a similarly situated test subject. For example, a first test subject may be compared against an average of a plurality of other test subjects that are of the same age and intelligence levels. Thereafter, once the comparison has been made of the test subjects' completion time, the system can provide a recommendation to a user, such as the teacher, to indicate that the test subject's attention and working memory is sufficient or falls below certain thresholds. Thereafter, the system can recommend an individualized educational plan or something similar thereto, to increase the test subject's working memory and attention in an effort to raise them up to minimum threshold standards or above.

In accordance with an aspect of the present disclosure, and in addition to providing real time brainwave neurofeedback to the test subject, the method and devices associated with implementing the same may provide a summary of brainwave feedback to the user in a mapped form subsequent to the conclusion of the activity or task. By providing a mapped form of the brainwave feedback data over the course of the task, the test subject is able to view and evaluate their brainwave data collectively over the entire task in a graphical format. Additionally, this mapped data may be sent to the central server in order to compare against a standardized rubric similar to the manner described above so as to enable various conclusions to be drawn or made by the system via an algorithm to determine whether the test subject has accomplished certain achievement thresholds. One exemplary standardization is to record data and upload it to central database for only the first ten minutes of a user's session. This enables all sessions of test users to be set or standardized to a fixed length so as to make the comparisons equal across multiple test subjects.

In accordance with another aspect of the present disclosure, the system may generate a system recommendation report in response to receiving raw information completion times per subject. Instructions encoded in a non-transitory computer readable storage medium may be executed by one or more processors to implement operations of evaluating the raw test data and providing a system recommendation to either a third party, such as a parent, or the user (such as a teacher). The system recommendations may include, but are not limited to, identifying whether the test subject was paying attention, identifying whether the test subject is increasing their working memory, identifying whether the test subject appears to be exceeding or failing to meet minimum threshold standards with respect to standardized rubrics or a standardized average of the students peers or other test subjects. The system recommendations assist the user or evaluator, such as the teacher, to compile the results in a dashboard format for easy viewing and graphical representation.

In accordance with one aspect of the present disclosure, an embodiment of the system and devices used to implement the brain training system may include a C-Sharp (C#) coded application program interface in order to connect the software implementing the two-phase attention and working memory tasks described above with the various hardware devices described herein. The API is required in order to integrate various tests and tasks into the system of the present disclosure. In accordance with another embodiment, the present disclosure provides an API for Neurosky hardware devices to integrate with C-Sharp program code.

In accordance with yet another aspect of the present disclosure, an embodiment may provide a brain training system, method, and device associated therewith that provides multi-modalities for providing feedback to the test subject during the implementation of the attention based task and the working memory based task. The multi-modalities may include and may relate to non-traditional methods or manners in which information is conveyed to the test subject. For example, additional ways of supplying data or information to the test subject may include lights, sounds, or other signals that trigger other senses of the test subject user. For example, a voice could be heard, a light can be seen, or another signal stimulating at least one of the other senses, such as haptic feedback.

In accordance with yet another aspect, an embodiment of the present disclosure may provide a method configured to improve brain functioning comprising the steps of: equipping a test subject with a neurofeedback monitoring device; providing a first task to be completed, wherein the first task is an attention training technique incorporating an image for identification; effectuating the completion of the first task; displaying, in real time, neurofeedback during the completion of the first task to the test subject; measuring a completion time of the first task and recording the completion time in a register; providing a second task to be completed, wherein the second task is a working memory training technique; effectuating the completion of the section task; displaying, in real time, neurofeedback during the completion of the second task to the test subject; measuring a completion time of the second task and recording the completion time in the register; comparing the completion times of the first and second task with predetermined standard completion times; providing a recommendation of attention and working memory of a test subject based on the comparison of the completion times of the first and second task with predetermined standard completion times, wherein if one of the completion times is less than the predetermined standard completion times then indicating one of the following (i) that the test subject is not improving attention span and (ii) that the test subject is not improving working memory, wherein if both the completion times are greater than or equal to the predetermined standard completion times then indicated that the test subject is increasing attention span and working memory. This exemplary method or another exemplary method may further provide wherein the step of effectuating the completion of the first task is accomplished over a first time frame in a range from one to three weeks. This exemplary method or another exemplary method may further provide wherein the step of effectuating the completion of the second task is accomplished over a second time frame in a range from one to three weeks. This exemplary method or another exemplary method may further provide wherein the step of effectuating the completion of the first task is accomplished over a first time frame in a range from 10 sessions to 30 sessions, wherein in each session is about 20 minutes in length. This exemplary method or another exemplary method may further provide wherein the step of effectuating the completion of the second task is accomplished over a second time frame in a range from 10 sessions to 20 sessions, wherein each session is about 20 minutes in length. This exemplary method or another exemplary method may further provide capturing and recording data from only a portion of the session to normalize results with other test subjects. This exemplary method or another exemplary method may further provide wherein only the results from the first ten minutes of a session is captured. This exemplary method or another exemplary method may further provide wherein the first task to be completed is identifying a match/mismatch between first and second images. This exemplary method or another exemplary method may further provide wherein the first and second images are displayed in a self-contained handheld device. This exemplary method or another exemplary method may further provide wherein the first and second images are displayed on a computer screen and a test subject identifies the match/mismatch via a mouse. This exemplary method or another exemplary method may further provide wherein the working memory training technique comprises the steps of: illuminating lights in a first sequence; receiving, via user input, repeated sequential illuminations; determining whether the repeated sequential illuminations matches the first sequence. This exemplary method or another exemplary method may further provide wherein the first sequence and the repeated sequential illuminations are accomplished entirely in a self-contained handheld device. This exemplary method or another exemplary method may further provide wherein the first sequence and the repeated sequential illuminations are accomplished in a computer application controlled via a mouse or a touch screen. This exemplary method or another exemplary method may further provide wherein the method is implemented in a school curriculum and the test subject is a school-age student. This exemplary method or another exemplary method may further provide wherein effectuating the completion of the first task comprises: generating, in a first round of testing, a first visual cue and a second visual cue simultaneously, wherein the first completion time begins being measured from the time the first and second visual cues are provided, wherein the first and second visual cues are one of the following: (i) a matched pair, and (ii) mismatched; receiving a response as to whether first and second cues are a matched pair or mismatched; determining whether the received response is correct, wherein if the response is correct then increasing difficulty in a second round of testing.

In yet another aspect, the present disclosure may provide a system comprising: at least one non-transitory computer readable storage medium having instructions encoded thereon, that when executed by one or more processors, perform operations to train attention and working memory in a test subject, the operations comprising: (i) conduct an attention-based task by presenting first and second images including geometric shapes, wherein the attention-based task requires identification, via user input, as to whether the first and second images match; (ii) conduct a working memory task by presenting sequenced illuminations, wherein the working memory task requires repetition, via user input, of sequenced illuminations; a display for displaying at least one of the attention based game/test and the working memory game/test; and at least one user input device in operative communication with the display. This exemplary system or another exemplary system may further include wherein the at least one user input device includes a first handheld controller, wherein the display is carried by the first handheld controller; and wherein the first display presents the first and second images during the attention-based task. This exemplary system or another exemplary system may further include wherein the working memory task is accomplished by the first handheld controller. This exemplary system or another exemplary system may further include a second handheld controller including at least one light for implementing the illumination sequence of the working memory task. This exemplary system or another exemplary system may further include at least one attention indicator including a plurality of lights adapted to be illuminated in response to neurofeedback information generated by the test subject; wherein the plurality of lights are illuminated in real-time so as to allow the test subject to visualize their attention during at least one of (i) the attention-based task and (ii) the working memory-based task, wherein the lights are positioned below an object shaped in the form of a human brain.

### BRIEF DESCRIPTION OF THE SEVERAL VIEWS OF THE DRAWINGS

A sample embodiment of the disclosure is set forth in the following description, is shown in the drawings and is particularly and distinctly pointed out and set forth in the appended claims. The accompanying drawings, which are fully incorporated herein and constitute a part of the specification, illustrate various examples, methods, and other example embodiments of various aspects of the disclosure. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 is a schematic view of an exemplary computing environment in which portions of the system and method of the present disclosure operate.

FIG. 2 is a schematic view of a general representation of a physical interaction model.

FIG. 3 is a schematic view of a general representation of a digital interaction model.

FIG. 4 is a schematic view of a general representation of a brain computer interface.

FIG. 5 is a schematic view of a system and method of the present disclosure depicting a first phase and a subsequent second phase coordinated in a manner to increase attention and working memory.

FIG. 6 is an exemplary screenshot view of an attention-based task or activity completed by a test subject in an application format displayed on a computer screen or monitor.

FIG. 7 is an exemplary screenshot view of a working memory-based task or activity completed by the test subject in an application format displayed on a computer screen or monitor.

FIG. 8 is a perspective view of exemplarily self-contained hardware device for implementing the attention-based task or activity.

FIG. 9 is schematic view of an exemplary self-contained hardware device for implementing the working memory-based task or activity and its associated operational parameters and relationship with software controls.

FIG. 10 is a perspective view of the exemplary self-contained hardware device for implementing the working memory-based task schematically depicted in FIG. 9.

FIG. 11 is a schematic timeline of the system and method for implementing phase one and phase two into a curriculum.

FIG. 12 is an exemplary flow chart depicting a method in accordance with one aspect of the present disclosure.

Similar numbers refer to similar parts throughout the drawings.

### DETAILED DESCRIPTION

The present disclosure relates generally to a method and system of a brain-computer interface (BCI) utilized to improve attention and working memory by implementing a two-phase approach.

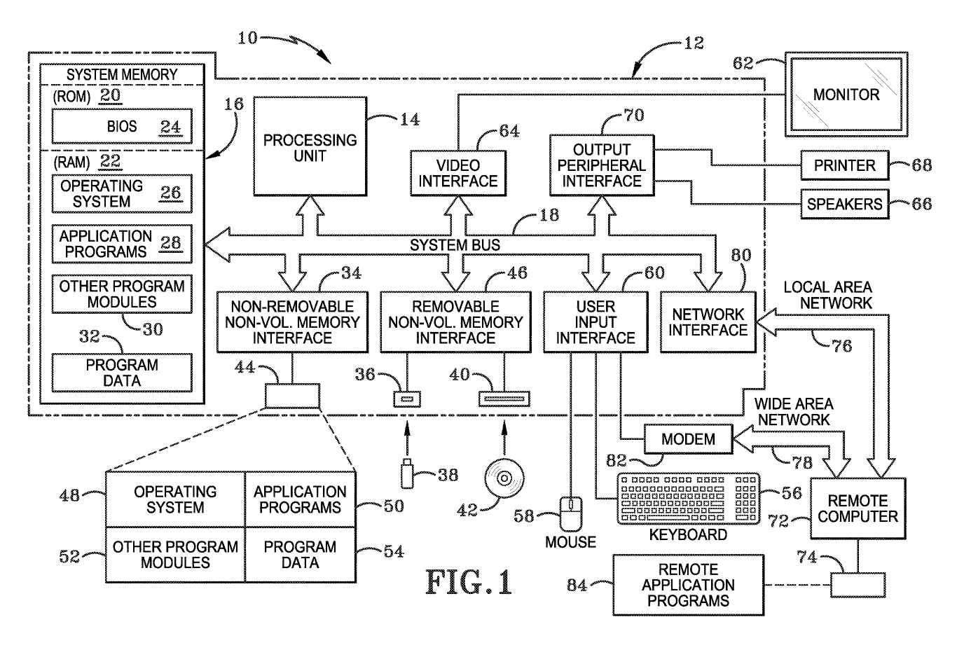

FIG. 1 depicts an exemplary computing system environment 10 which can define one half of the BCI (the other half being the test subject's brain to which no claim of invention is made to the human element). The computing system environment 10 on which the claimed method and programmed memory and apparatus may be implemented. The computing system environment 10 is only one example of a suitable computing environment and is not intended to suggest any limitation as to the scope of use or functionality of the invention. Neither should the computing environment 10 be interpreted as having any dependency or requirement relating to any one or combination of components illustrated in the exemplary operating environment 10.

The claimed methods, programmed memory and apparatus are operational with numerous other general purpose or spatial purpose computing system environments or configurations. Examples of well-known computing systems, environments, and/or configurations that may be suitable for use with the invention include, but are not limited to, personal computers, server computers, hand-held or laptop devices, smart phones (such as an Apple iPhone or a Samsung Galaxy or the like), multiprocessor systems, microprocessor-based systems, set top boxes, programmable consumer electronics, network PCs, minicomputers, mainframe computers, distributed computing environments that include any of the above systems or devices, and the like.

The claimed methods, apparatus and programmed memory may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 1, an exemplary system for implementing the claimed methods, apparatus and programmed memory includes a general purpose computing device in the form of a computer 12. Components of computer 12 may include, but are not limited to, a processing unit 14, a system memory 16, and a system bus 18 that couples various system components including the system memory to the processing unit 14. The system bus 18 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 12 typically includes a variety of computer readable media. Computer readable media can be any available media that can be accessed by computer 12 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. This may also include non-transitory computer readable storage media. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 16 includes computer storage media in the form of volatile and/or nonvolatile memory such as read only memory (ROM) 20 and random access memory (RAM) 22. A basic input/output system 24 (BIOS), including the basic routines that help to transfer information between elements within computer 12, such as during start-up, is typically stored in ROM 20. RAM 22 typically includes data and/or program modules that are immediately accessible to and/or presently being operated on by processing unit 14. By way of example, and not limitation, FIG. 1 illustrates operating system 26, application programs 28, other program modules 30, and program data 32.

The computer 12 may also include other removable/non-removable, volatile/nonvolatile computer storage media. By way of example only, FIG. 1 illustrates a hard disk drive 34 that reads from or writes to non-removable, nonvolatile magnetic media, a drive 36 that reads from or writes to a removable, nonvolatile magnetic disk or USB flash drive 38, and an optical disk drive 40 that reads from or writes to a removable, nonvolatile optical disk 42 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 44 is typically connected to the system bus 18 through a non-removable memory interface such as interface 34, and drive 36 and optical disk drive 40 are typically connected to the system bus 18 by a memory interface, such as interface 46.

The drives and their associated computer storage media discussed above and illustrated in FIG. 1, provide storage of computer readable instructions, data structures, program modules and other data for the computer 12. In FIG. 1, for example, hard disk drive 44 is illustrated as storing operating system 48, application programs 50, other program modules 52, and program data 54. Note that these components can either be the same as or different from operating system 26, application programs 28, other program modules 30, and program data 32. Operating system 48, application programs 50, other program modules 52, and program data 54 are given different numbers here to illustrate that, at a minimum, they are different copies.

A user may enter commands and information into the computer 12 through input devices such as a keyboard 56 and pointing device 58, commonly referred to as a mouse, trackball or touch pad. Other input devices (not shown) may include a touchscreen, buttons, individual keys, microphone, joystick, game pad, satellite dish, scanner, or the like. These and other input devices are often connected to the processing unit 14 through a user input interface 60 that is coupled to the system bus 18, but may be connected by other interface and bus structures, such as a parallel port, game port or a universal serial bus (USB). A monitor 62 or other type of display device is also connected to the system bus 18 via an interface, such as a video interface 64. In addition to the monitor 62, computers may also include other peripheral output devices such as speakers 66 and printer 68, which may be connected through an output peripheral interface 70.

The computer 12 may operate in a networked environment using logical connections to one or more remote computers, such as a remote computer 72. The remote computer 72 may be a personal computer, a server, a router, a network PC, a peer device, a smart phone or other common network node, and typically includes many or all of the elements described above relative to the computer 12, although only a memory storage device 74 has been illustrated in FIG. 1. The logical connections depicted in FIG. 1 include a local area network (LAN) 76 and a wide area network (WAN) 78, but may also include other networks. Such networking environments are commonplace in offices, enterprise-wide computer networks, intranets and the Internet.

When used in a LAN networking environment, the computer 12 is connected to the LAN 76 through a network interface or adapter 80. When used in a WAN networking environment, the computer 12 typically includes a modem 82 or other means for establishing communications over the WAN 78, such as the Internet. The modem 82, which may be internal or external, may be connected to the system bus 18 via the user input interface 60, or other appropriate mechanism. In a networked environment, program modules depicted relative to the computer 12, or portions thereof, may be stored in the remote memory storage device. By way of example, and not limitation, FIG. 1 illustrates remote application programs 84 as residing on memory device 74. It will be appreciated that the network connections shown are exemplary and other means of establishing a communications link between the computers may be used.

Now that computing environment 10 has been described, reference is made to the interactive system, method, and article of manufacture for training a brain of the present disclosure, to which the computing environment 10 helps implement.

The present disclosure relates to a two-phase system for increasing attention (phase one) and working memory (phase two) in test subjects by selectively training the brain. The method of training the brain of present disclosure may be implemented in multiple ways. The two phases of the system of the present disclosure may be implemented in either a hardware device that the test subject (i.e., student) can pick up and hold, or may be implemented in an application run on a computer and displayed in a monitor, such as monitor 62, that the test subject interacts with.

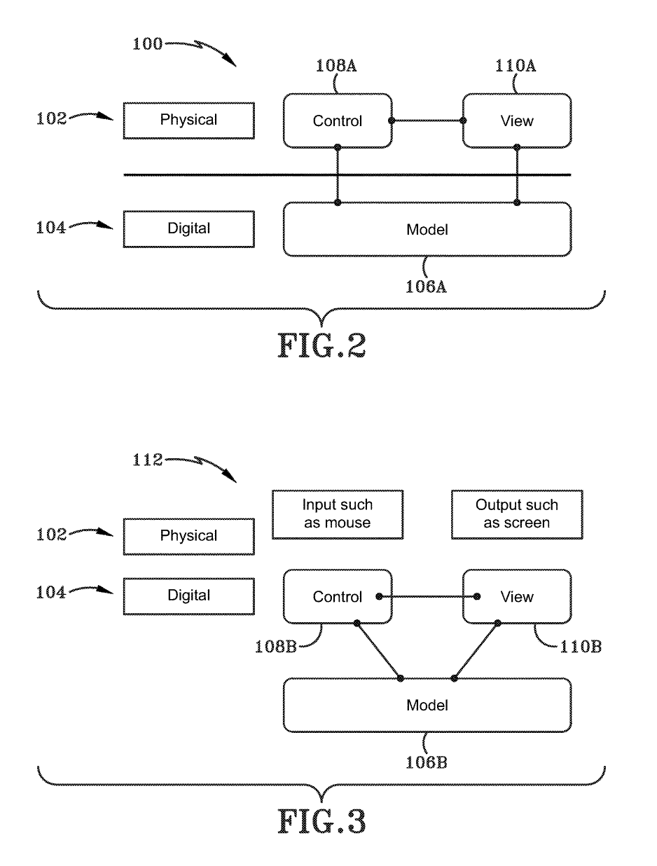

FIG. 2 is a schematic view of a general representation of a physical interaction model (such as when the test subject is interacting with a physical device implementing the two phase system of the present disclosure). The physical interaction model 100 delineates between a physical realm 102 and a digital realm 104. A digital implementation model 106A exists in the digital realm 104, whereas the control unit 108A exists in the physical realm 102. Additionally, the viewing device 110A exists in the physical realm 102. The control unit 108 may be a handheld device, such as a tablet-like device with control buttons integrated therewith. The tablet-like device enables the test subject to interact with the device. Furthermore, there may be two devices (one for each phase), or each phase could be implemented on a single tablet-like device.

Interaction is the dialog between the test subject and the computer 12, while the interface is the vehicle for that dialog. In the field of human computer interaction, the capacity to provide user (i.e., the test subject) control over the interface is a vital component and provides users with comfort in using the interface. Some exemplary properties of tangible user interfaces are physical interaction, rich feedback, and high levels of practicality. There has been an increase in tangible user interfaces for complex real-world application domains such as learning and health. When designing a tangible physical manipulation interface, the user should feel as if he or she is interacting with the domain rather than with a computer interface, so the user focuses on the tasks instead of the technology.

In mathematics, tangible teaching instruments (e.g., blocks, rods, board games) can reinforce mathematical understanding of numbers, lines, and other mathematical entities. These instruments can give learners ways to construct physical models of abstract mathematical ideas. When children learn by using an instrument, information is encoded in a different neural location than when they learn by methodology. To design an interface that is effective in supporting children's learning, it is important to understand the limitations and advantages of the physical directive manipulation. The tangible designs may support learning of mathematics by building on the advantages of physical manipulation, while avoiding memorization of mathematical theories, which supports ongoing research into children's use of physical materials to solve numerical problems, and comparative performance using virtual materials. Physical manipulation interfaces are seen as likely candidates to influence advanced user interfaces. User control and responsibility are highly desirable. Traditional computer interfaces frame interaction in terms of “input” and “output”. The computer output is delivered in the form of “digital illustrations”, while the input is obtained from controls such as keyboard and mouse.

FIG. 3 is a schematic view of digital interaction model 112 depicting the relationship between the viewing device 110B and control unit 108B in a traditional application-based graphical approach. A digital implementation model 106B exists in the digital realm 104. Similarly, the control unit 108B exists in the digital realm 104. Additionally, the viewing device 110B exists in the digital realm 104. The control unit 108B may be a computer monitor that is control by a computer mouse or touchpad. The traditional graphic user interfaces highlight the strong separation between the view provided by the display and the control provided by the mouse.

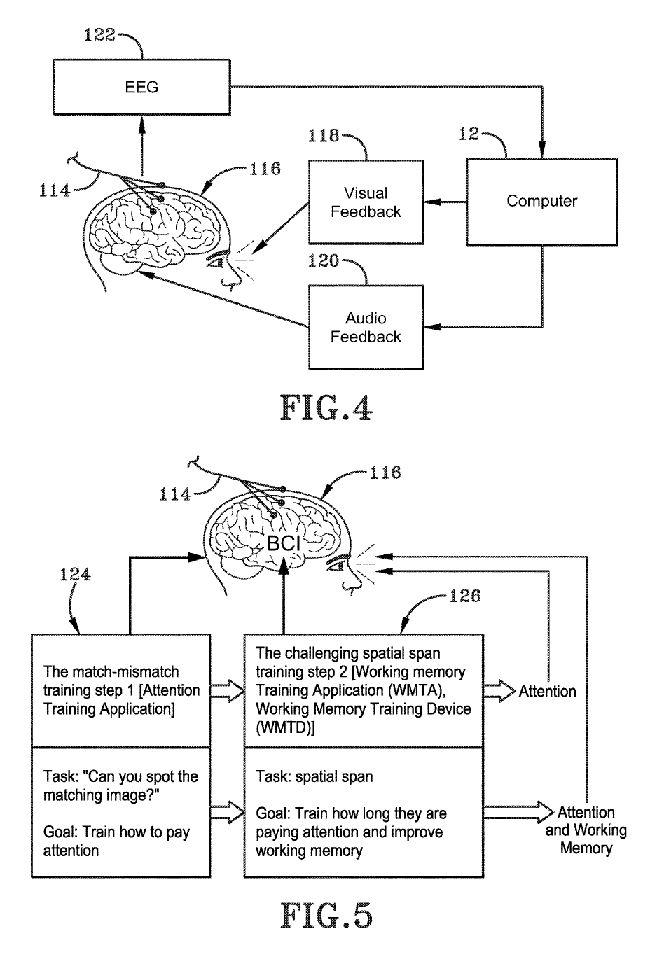

New FIG. 4 is a schematic of a neurofeedback principle. An exemplary neurofeedback capturing device 114 is work around the test subject 116 head. Device 114 is linked with computer 12 so as to allow neurofeedback output from the device to be input into computer 12 and operatively connected with system bus 18 for processing by processing unit 14. The neurofeedback capturing device 114 is in electrical communication with the computer 12 which drives visual cues or feedback 118 and audio cues or feedback 120. As the test subject interprets and senses the visual and audio cues or feedback 118, 120, brain activity is recognized by the capturing device 114. In EEG neurofeedback training programs, the participant wears an EEG net (i.e., capturing device 114) and views computer feedback of his or her brain activity 122. The participant is encouraged to learn to “control” this readout by receiving coaching from a clinician on maintaining effort and focus using metacognitive strategies. Users learn to generate specific brainwaves through various mental strategies while monitoring the outcome of their efforts in near real time.

FIG. 5 schematically depicts phase one 124 and phase two 126 of the present disclosure, which may also be referred to as a first step or task or activity and a second step or task or activity respectively. The system may include a software application and/or hardware direct manipulative interface device, such as a physical memory manipulative device (ATD, WMTD) and and/or a non-physical memory manipulative application (ATA, WMTA). As used herein the acronym “ATD” refers to an attention-based physical memory manipulative device for completing the attention based task (i.e., phase one). The acronym “WMTD” refers to a working memory-based physical memory manipulative device for completing the working memory-based task (i.e., phase two). The acronym “ATA” refers to an attention-based non-physical memory application (i.e., such as on computer 12) for completing the attention based task (i.e., phase one). The acronym “WMTA” refers to an working memory-based non-physical memory application (i.e., such as on computer 12) for completing the working memory-based task (i.e., phase two). Notably, the present disclosure relates to the system and method for training the brain by first completing the attention-based task, then thereafter completing the working memory based task. However, their respective implementations may be accomplished in either a physical device or a software application.

In one example, a matching task is executed from instructions encoded on a non-transitory computer readable storage medium, that when executed by at least one processor, performs an operation of training subjects on how to pay attention and focus on the task at hand (Phase one 124). The spatial-span step or task (phase two 126) is executed from instructions encoded on a non-transitory computer readable storage medium, that when executed by at least one processor, performs an operation of training subjects to view how long they are paying attention. The goal was to enhance working memory capacity primarily by increasing the amount of visuospatial information to be retained and by observing attention feedback on the screen (non-physical memory brain training matching application) or the LEDs (physical memory-brain training matching application).

During the matching task (phase one 124), a new task appears on the screen showing two matched or mismatched images. The subject makes a choice to press the match or mismatch button. Time to complete the task is measured from the beginning to the successful completion of the task. When the correct answer is displayed, a green LED lights up and the level for the next task increases by one. The score for the task is also increased by one and the next task begins.

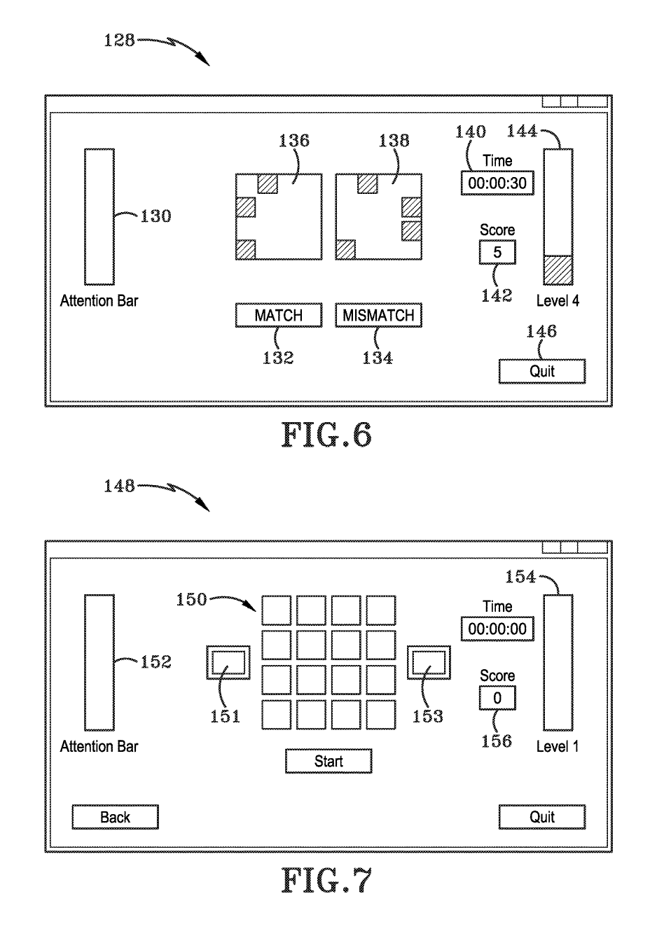

FIG. 6 and FIG. 7 represent exemplary application-based (i.e., not physical) embodiment of the a brain training system in accordance with the present disclosure.

FIG. 6 depicts an exemplary screen shot of the ATA (attention-based application) 128 including an Attention Bar (Brain wave Feedback) 130, a match button 132, a mismatch button 134, a first complex image 136, a second complex image 138, a timer 140, a score indicator 142, a level of difficulty indicator 144, and a quit button 146.

The first complex image 136 includes at least one geometric shape, such as a square. Further, the second complex image 138 includes at least one geometric shape, such as a square. Further, in each complex image, the geometric shapes are associated with a specific location. For example, the first and second complex images may generally utilize a 4×4 array wherein each box of the array (16 boxes total) can be illuminated or highlighted to form a geometric square. Thus, when a box of the array is selected, it forms a shape which the test subject identifies relative to other portions of the array. Thus, in addition to each complex image having geometric shapes, the shapes are in specific locations of the image. Thus, when the test subject must identify whether the first and second complex images are “matched,” the test subject must identify the correct number of geometric shapes as well as the correct location/positioning of the shapes in each respective image. In one example, it may be considered critical that the complex images are formed from geometric shapes, and not other deigns or figures, such as faces or geographic landscapes. The present disclosure has found, through empirical evidence that geometric shapes test more effectively to increase attention over other designs or figures, such as faces.

The attention bar 130 is linked with the neurofeedback device 114 worn on the test subject's 116 head. As the computer 12 receives neurofeedback input from the device 130 worn on the head, it is display in real-time (without perceivable delay to the test subject) on the display in the form of the attention bar 130.

If the test subject 116 identifies a correct match, the level indicator 144 increases and the complexity of the first and second images increases. For example, in the first round, the first and second images may have one, two or three geometric shapes to identify a match/mismatch. However, in the second round, the first and second images may have three, four, or five geometric shaped to identify a match/mismatch. The task of identifying a match or mismatch of first and second complex images is an attention-based task (phase one) that task requires the user to concentrate or focus attention on complex images. Thus, at the frequency of completing phase one continues, the test subject should be able to improve their attention ability. Furthermore, the period of phase one may be considered critical to the functionality of the system in some implementation, for example, in one embodiment, the test subject should perform the attention-based task for frequency at least once a day for at least 10 minutes at a time, for a period of at least two weeks.

With respect to FIG. 6, the subject has the choice of pressing on the match button 132 or mismatch button 134. Times are measured at the beginning of the task and the end of the task via timer 140. If answer is correct, a green LED turns on and the level increases by one and the score increase by 1 (and the next task starts). A new task appears on the screen showing the two mismatch images. The subject has the choice of pressing on the match or mismatch buttons. Times are measured at the beginning of the task and the end of the task. If answer is incorrect, a red LED turns on and the level decreases by one, so the next task starts with a fewer quantity of boxes (less complex image). A new task appears on the screen shows the two mismatch images. The subject has the choice of pressing on the match or mismatch buttons. Times are measured at the beginning of the task and the end of the task. If subject answers two tasks correctly, the score increases by 2, the green LED turns on and the next task starts (more complex image).

For each round, a new task with a new set of possible solutions to the task appears. In one embodiment, the images for each round are randomly generated each time, however the code/instructions may indicate that about 50% of the time the images should match, but how they match/what patterns the form to create the match may be random. Since humans are selective in focus of attention, in this step, children were asked to identify whether the images were matched or mismatched.

Subject(s) 116 make a selection of either match/mismatch using any of the interfaces (either 1. hardware based handheld controller or 2. the application based computer game/program). A similar algorithm or set of instructions is embedded with the application to control the LEDs on or off in the physical handheld controller interface and the attention bar and the level bar in the application-based interface. The algorithm measures and records attention and other EEG waves into the database.

In one non-limiting example, the test subject 116 is a student in an elementary school class room, the subject 116 is called from the classroom to attend the training in an isolated area in the library. The subject dons and continues to wear the NeuroSky MindSet (i.e. the neurofeedback device 114) that forwards brain wave signals to the software application and logs into the application using a unique username and password executed via processing unit on a computer, such as computer 12 or the like. Upon signing in, the application recognizes the sign in role as a subject or a teacher. A welcome message then appears on the screen and the subject clicks “Start” to begin the session. Two images appear on the computer screen if the subject is using the ATA (Attention Training Application) 128 or on the LCD if using the ATD (handheld Attention Training Device, described in greater detail below) and training begins. The timer 140 is used to document the total amount of time the subject is engaged in each task, with brain computer interface readings captured every second, for example. The software application calculates the average attention every second and records the average attention information into a database (Recommended average reading to really capture an action pattern). The classification system may continuously analyze the incoming brain waves, which are mapped at the conclusion of the subject's session. Student actions control some feature(s) of the running session. The timer captures the amount of time the students spends in each learning session. The amount of time per session is not limited as students can participate as long as they chose. However in one example, the data analysis only captures the first 10 minutes of each session to normalize the session with other test subjects who may have a period of time more than 10 minutes to play the tasks/games. The subject can view their amount of time in each session, level, and score at all times during the training session. Students make their selections by clicking on the buttons to match or mismatch if ATA or by clicking on the yellow or the green push button and in the ATD to indicate match or mismatch. A new task appears as soon as the student completes the current task and the task completion time is recorded to measure the difference between the ending task time and beginning task time. The application automatically calculates the level of each task. If the subject gets two tasks in a row correctly, it automatically moves to the next level. Subjects receive 1 point for each correct answer.

FIG. 7 depicts an exemplary screen configured to implement the working memory-based application (WMTA) 148 task of the brain training system (i.e., phase two 126) in accordance with the present disclosure. The working memory-based application task 148 (i.e., phase two) may include a first sequentially illuminated light sequence on lights 150, a second light sequence to be repeatably sequentially entered by touching lights 50, via user input, to be displayed on an screen or display inherently via the lights, a brain wave indicator 152, a progress (or difficulty level) indicator 154, a correct answer indicator 151, and an incorrect answer indicator 153, and score indicator 156.

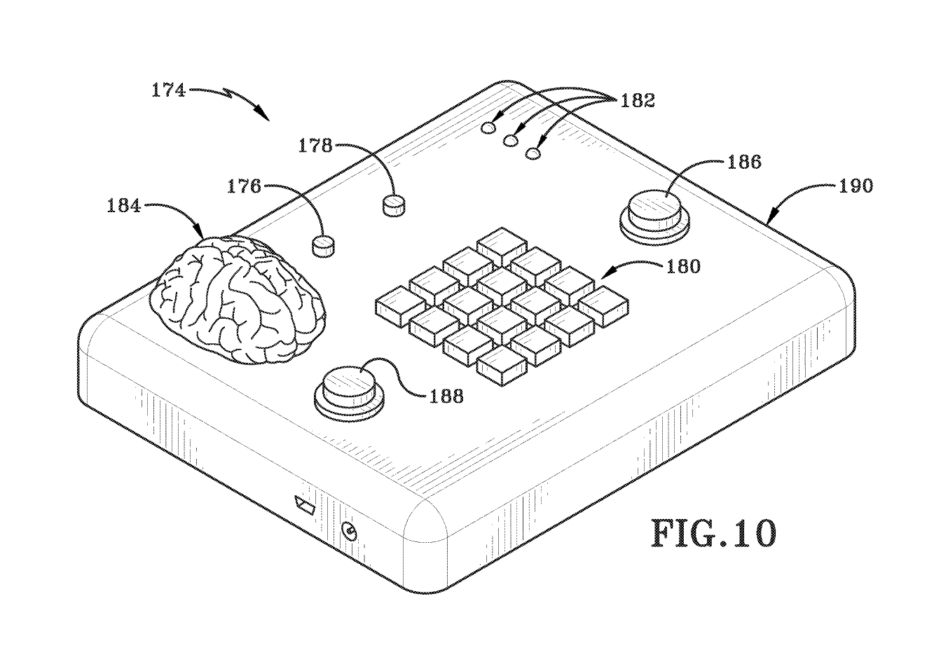

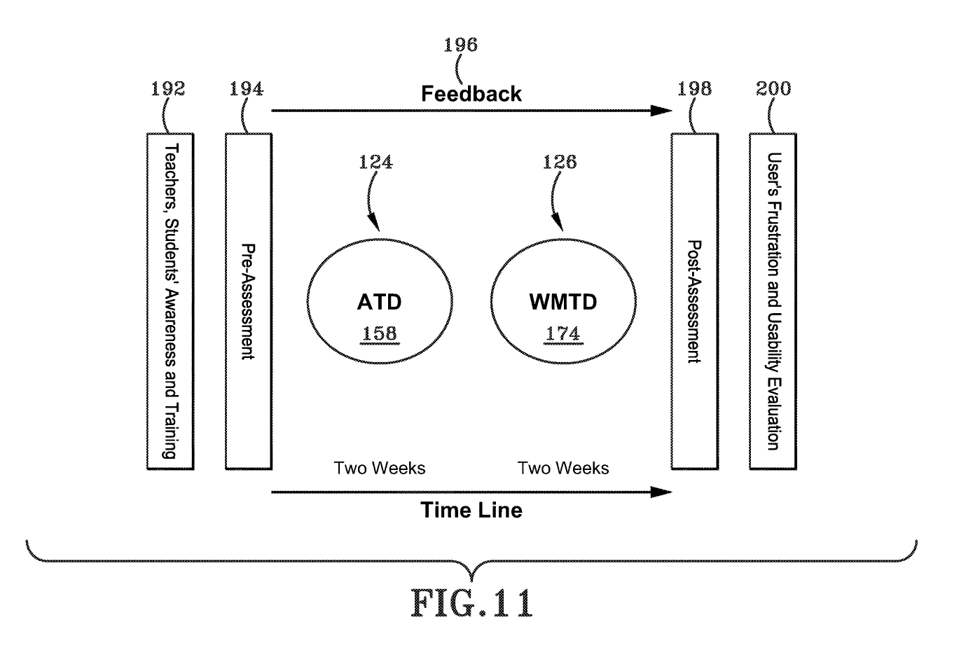

The brainwave feedback indicator 152 is generally indicated in FIG. 7 as a bar or strip of lights, such as blue lights on the display screen. However, other embodiment may incorporated these feedback indicators into a visual representation a human brain (similar to the physical device as shown in FIG. 10), wherein the lights are positioned within the brain to represent areas of the brain that are activated (as observed by the neurofeedback device) in real time.

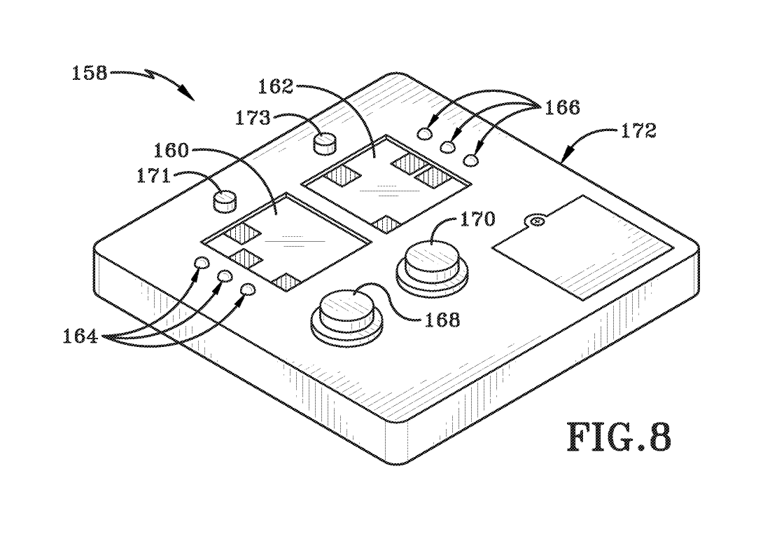

FIG. 8 depicts an exemplary ATD hardware device 158 that accomplishes a similar goal as the ATA 128 identified in FIG. 6. The hardware ATD device 158 used to accomplish the attention-based task/activity includes a first sequentially illuminated image 160, a second image 162 to be identified, via user input, of a match/mismatch of the image, a brain wave and attention indicator 164, a progress indicator 166, a match indicator 168, and a mismatch indicator 170, a correct answer indicator 171, and an incorrect answer indicator 173 all contained in a self-contained unit or assembly. The interface shown in FIG. 8 demonstrates the push buttons for selection of the match indicator 168 or mismatched indicator 170 that the child (or test subject) uses. The three yellow LEDs (i.e., correct answer indicator or performance/progress indicator 166) indicate the child's performance during the activity while the three blue LEDs measure (i.e., attention indicator 164) and indicate the child's attention level. The Arduino Leonardo is a microcontroller board based on the Atmega328 that is programmed using the Arduino IDE. A liquid crystal display (LCD) screen is utilized to display the task on the screen and all of the components are stored in a box or housing 172.

FIG. 8 depicts an example of an Attention Training Device (ATD) which is a substantially hardware based, tablet-like device configured to be self-contained and held by the test subject. The ATD may be include a non-transitory computer readable storage medium therein having instructions encoded thereon configured to perform the aforementioned operations of the Attention-based task/activity of identifying matching/mismatched geometric shapes.

With respect to the attention-based activity/task, each of the ATA and the ATD calculate the level per session, if the test subject answers correctly for two tasks in the row, then they receive 2 points and if they answer only one they get 1 points. Similarly, if the student answers incorrectly, the instructions are encoded to deduct 1 point.

The system may include a software application and/or hardware direct manipulative interface device, such as a physical memory manipulative device (ATD, WMTD) and and/or a non-physical memory manipulative (ATA, WMTA).

Three levels of attention threshold are defined in the application as shown in Table 1 and Table 2. While the three levels of attention presented in Table 1 and Table 2 are exemplary, the present disclosure is not limited to the three levels of attention. Other levels could be implemented, for example five levels or ten levels. In the example wherein the attention bar on either the attend-based device or application and the attention bar on either the working memory based-deice or application utilizes LEDs or other lights, one LED or light corresponds to one level. So for example, if the system divides the attention threshold into three levels, then the attention bar consists of three LEDs (one for each level). If the system divides the attention threshold into five levels, then the attention bar consists of five LEDs (one for each level). If the system divides the attention threshold into ten levels, then the attention bar consists of ten LEDs (one for each level), and so on.

In each instance, the subject 116 wears a noninvasive brain computer interface 114 around their head. For each task level, the subject is challenged to reach the highest attention level (i.e. the third level, or fifth level, or tenth level depending on the total number of LEDs). The attention indicator, which may be a LED strip bar, increases based on the level of attention.

TABLE 1
The Attention Level and Visual feedback

| Average | Visual Feedback | |
|---|---|---|
| Attention | Type | Action |
| 0 to 30 | LED LEVEL 1 | Turn On |
| >=31 and <=60 | LED LEVEL 2 | Turn On |
| >=61 and <=100 | LED LEVEL 3 | Turn On |

With respect to attention of the test subject, the present disclosure attempts to maximize Beta waves (>13 Hz) and minimize Theta waves. As the subject completes the attention based task, either with the ATA or ATD, the neurofeedback device worn around the head registers the brain wave frequencies (identified below in Table 2). As the number of Brainwaves in the Beta range are maximized, the threshold levels identified in Table 1 are registered in the systems (either in the device or the computer) and control logic illuminates the appropriate LED or light to identify the attention level.

TABLE 2
Brain wave frequencies and associated mental states

| Type | Hz = Cycles Per Second | | |
|---|---|---|---|
| Delta | <4 | Hz | Sleep, unconscious; |
| | | | increased in some ADHD, |
| | | | normal or decreased in |
| | | | others |
| Theta | 4-7 | Hz | Drowsiness, unfocused; |
| | | | increased in frontal and |
| | | | central area of brain in |
| | | | ADHD, continues into |
| | | | adulthood |
| Alpha | 8-12 | Hz | Eyes closed, relaxed, but |
| | | | alert; mixed findings, |
| | | | perhaps depending on age |
| | | | and gender |
| Beta | >13 | Hz | Mental activity, |
| | | | concentration; decreased in |
| | | | some but not all ADHD |
| | | | children, may normalize in |
| | | | adults |

As the test subject continues to complete the attention-based task (identify the match/mismatch), a performance indicator (which may be separate and distinct from the attention indicator) can identify the number of correct answers in a row. Three levels of performance threshold are defined in the application indicating the number of times the subject reaches each level, time, and errors (See Table 3 below). The subject uses a touchscreen, trackpad, or mouse to interact with the system when implemented in an application based environment. Alternatively, the subject interacts with a physical button when the brain training system of the present disclosure is implemented in a hardware based environment (i.e., a handheld tablet-like device).

TABLE 3
Performance Levels and Visual Feedback

| Correct Answers | Visual feedback type | Action |
|---|---|---|
| 0 to 30 | LED Level 1 | Turn On |
| >=31 and <=60 | LED Level 2 | Turn On |
| >=61 | LED Level 3 | Turn On |

The Neurosky application-programming interface is developed to integrate the brain computer interface with software (which may be coded in for example the C# paradigm programming language). The brain computer interface calculates the subject's attention and generates a reading from 0 to 100 during every second of real time with the average response of the headset recorded in the database. An attention threshold is calculated (e.g., Table 1 and Table 3) to control the blue LEDs of the attention indicator in the ATD interface and the attention indicator in the ATA.

In some example embodiments, there is provided, as noted, a child-friendly physical memory brain training system. The system is configured to have a positive effect on a child's engagement and performance, leading to an increase in attention. The system may include a software application and/or hardware direct manipulative interface device, such as a physical memory manipulative device (ATD {for attention-based tasks} and WMTD {for working memory-based tasks}) and/or a non-physical memory manipulative (ATA {for attention-based tasks} and WMTA {for working memory-based tasks}).

Phase two 126 of the present disclosure relates to a working memory-based task which occurs subsequent to the attention based task identified above. It has been empirically discovered that the frequency and period of performing phase on (attention-based task) prior to phase two (working memory-based tasks may be critical in improving overall brain function).

In some example embodiments, a software application (i.e., the WMTA 148) and a hardware physical memory application (i.e., the WMTD 174) are provided to effectuate a working memory-task/activity based on the spatial-span concept. The spatial-span concept is a tool used in clinical neuropsychology to exercise visuospatial working memory. Neuroscience studies have shown that visuospatial memory can be improved through “chunking” strategies. This is another way to take advantage of meaningful knowledge that one already possesses to facilitate retrieval and enhance working memory. In this task, the subject must remember the pattern of the lighted squares and repeat it by using the interface to interact with the application.

With respect to phase two 126, the subject 116 may have to remember the pattern of the lighted squares and repeat it by using the interface to interact with the application. In one example, there are 16 levels of illumination patters.

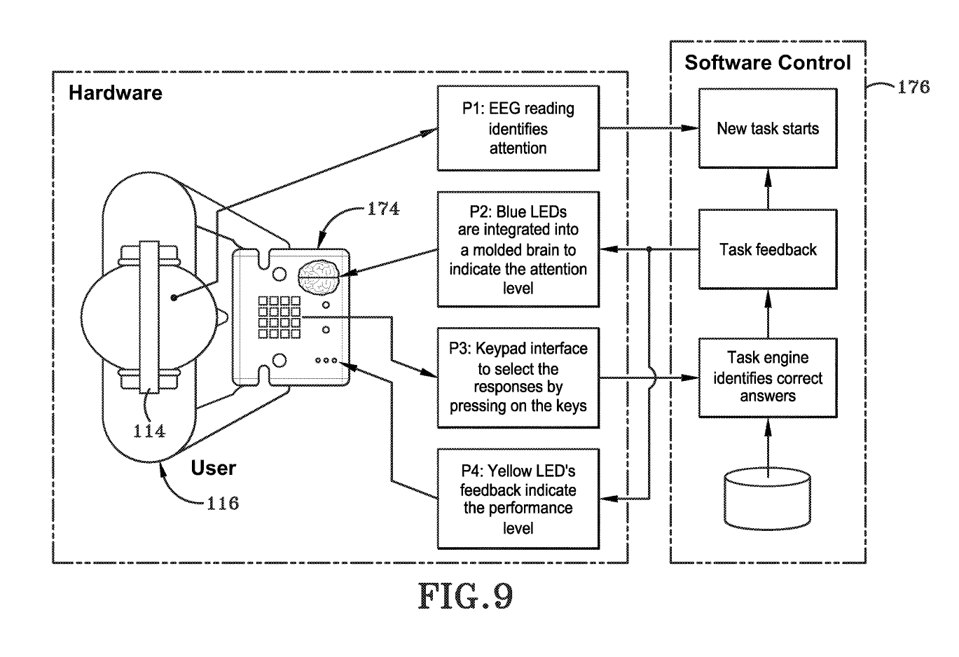

As depicted in FIG. 9 and by way of non-limiting example, some exemplary steps of the phase two working memory process include the subject get called form the classroom to attend the training in an isolated area in the library. A subject is wearing the NeuroSky mindset device 114 that forwards brain wave signals to the software control. This information will then be used to train a classification system so it can learn to recognize and thus map different brain patterns to actions. The subject 116 may then login to the system using a unique username and password. The system recognizes the sign in role as a student or a teacher. A welcome message appears on the screen. The subject clicks on a button for training to start. Two images appear on the screen if using the WMTA and on the LCD if using the WMTD. A timer is reading several readings from the BCI every second, and the system captures and read the attention per second. The application calculates the average attention every 15 second and recorded into the database, the classification system will continuously analyze the incoming brain waves and map them to the display indicator. Appropriate actions and thus control some feature(s) of the running session, in the WMTA case is the attention bar and the WMTD is the blue LED on/off according. The timer (or a second timer) captures the session time (for the data analysis empirical testing only captured the first 10 minutes). The test subjects have are facing the application and monitoring their attention and level progress while they are working on the training task. In one implementation, then, one of the keypad buttons lights up (WMTD) or the buttons lights up (WMTA). A timer is set for few seconds (such as 5 seconds). The buttons or keys turn off and the test subject has to remember and recreate the process by pressing on the physical keys (WMTD) or buttons on display (WMTA) in the correct order. The task completion time is recorded to measure the difference between the ending task time and beginning task time. They make their selection by clicking on the buttons to match or mismatch if WMTA or by clicking on the yellow or the green push button or select match or mismatch. This spatial-scan task of FIG. 9 is based on a number of items most people can remember is in a range between 2 and 7. Thus, typically the first round starts with only two buttons lighting up in sequence by can go up to 16 or 17 levels of sequentially illuminated buttons, but these would be very difficult.

FIG. 9 is a schematic view of the working memory task (i.e., phase two) implemented in the hardware device (WMTD). The interfaces enables student interaction with multiple input/output modalities are integrated in the design—tactile, brain activity as an input and visual feedback, brain activity on the screen, and a LED strip as an output. The user wears a noninvasive BCI that is encoded with the software application to provide additional modality to the physical manipulation-brain training system. In this implementation, the LED strip colors are controlled by the subject's average attention and performance. Subjects can increase their (attention and WM capacity) brainwaves (beta) and suppress drowsiness brainwaves (theta) by focusing on the task to try to achieve higher performance. In the WMTD 174 brain system, subjects are trained to enhance their visuospatial WM by increasing the number of retained items. The subject attempts to remember the pattern of the lighted squares and repeat it by using the interface to interact with the application. There are 16 levels of LED patterns with the number of lighted squares increasing with each level. The subject uses the software to complete the “remembered squares”.

The brain computer interface may connect to a computer via a wired and/or wireless link, such as a Bluetooth link. The MindSet brain computer interface uses Bluetooth to transmit data. The manufacturer designed the headset to be adjustable for various sizes of heads. The forehead sensor arm is designed to fit the forehead comfortably; however, in the current study it was difficult to adjust the arm to fit each child's head. An ordered headset size reduction did not work, so a simple modification to the sensor's position was made by adding an ear clip for better contact with the sensors.

FIG. 10 depicts the working memory-training interface (WMTD 174). The WMTD 174 includes an illuminable correct answer indicator 176, an illuminable incorrect answer indicator, an array of lights 180, a progress indicator 182, and a neurofeedback indicator 184, a start button 186, and a quit button 188. FIG. 10 depicts the WMTD 174 of the present disclosure as a self-contained tablet-like device for accomplishing the working memory task. The interface for this application is shown in FIG. 10 and includes the selection button pad 4×4 of lights 180 which define the interface and are made of a translucent silicon rubber button pad with 16 buttons. The idea is that the user creates a button interface of choice with the ability to display simple color under each button when the user presses it. The button has a very nice tactile feel. The Adafruit 4×4 Trellis printed circuit board (PCB) keypad is an open source backlit keypad driver system. Three yellow LEDs are utilized to indicate the child's performance on the activity and three blue LEDs are utilized to indicate attention, while an LCD screen displays the task status. An Arduino Mega microcontroller is used to design the selection button pad based on the Atmega1280. A 3D case for the 4×4 selection keypad (i.e. lights 180) was designed and printed using a 3D printer. All of the components are stored in a box or housing 190.

An exemplary feedback of the present disclosure provides a the WMTD device (or the ATD device) which actively incorporated brain neurofeedback of working memory (or attention, as the case may be) in a three dimensional (3D) object, such as the neurofeedback indicator 184. More particularly, the reference to the WMTD and the ATD being “three dimensional” refers to the ability of the test subject to hold the device in their hand and control the object (as opposed to simply interacting with a computer screen). In one implementation, the three dimensional devices. Furthermore, the three dimensional aspect of the present disclosure further includes the keypad on the WMTD to which the test subject interacts with. More particularly, the keypad is formed from a tactilely-pleasing substance so as to stimulate the test subjects sense of touch, as well as their sense of pressure. The keypad is housed and sized to receive lights 180 there beneath. The keypad may be at least partially transparent or translucent to allow the illuminations of the lights 180 beneath the keypad to light up during the course of the repetitive working memory-based activity.

The neurofeedback indicator 184 may be a three dimensional object shaped in the form of a human brain. When in the shape of a human brain, the indicator 184 rises above the housing 190 in a prominent manner. The indicator 184 may include a plurality of lights positioned beneath the outer surface of the brain shaped object. These lights are in operative communication 12 with the processor that receives brainwave feedback from the device 114 worn by the test subject 116. In this way, the test subject 116 can see their brain activity in real-time in the lights that are positioned in the brain of the indicator 184. The indicator must be at least partially transparent or translucent to enable light from the lights positioned below the brain shaped object to illuminate in real time so as to provide accurate neurofeedback for the test subject as to their real-time (without perceivable delay to the user) brain activity.

The brain training system of the present disclosure may further comprises a central computer or central database, which may operate within computing environment 10 so as to be considered a second computer 12. In this instance, the central computer stores and registers in a database all data sets of test participants.

A database program executed by at least one processor of the central processor processes the information on a per test subject basis and can compare each individual test subject to a set of averages (either mean, medians, or modes) of other test subjects. The central server may then provide a recommendation of attention and working memory of a test subject based on the comparison of the completion times of the first and second activity/task with either predetermined standard completion times or average completion times, wherein if one of the completion times is less than the predetermined standard completion time or average of the remaining test subjects then indicating one of the following (i) that the test subject is not improving attention span and (ii) that the test subject is not improving working memory, wherein if both the completion times are greater than or equal to the predetermined standard completion times then indicated that the test subject is increasing attention span and working memory. This may be generally referred to as a “system recommendation.”

With continued reference to the system recommendations, some exemplary system recommendations may recommend to the teacher or parent after the student has completed either one training session or the entire course. In one example, the system will generate a recommendation report to the parents or teachers. The report may include quantitative data and graphs. The data and graphs may comprise the average attention span of the test subject 116 while working on a task during a pre-assessment phase (i.e., before the first training exercise or phase one 124). The data and graphs may further comprise the attention span of the test subject 116 while working on a task after completing the first training exercise or phase one 124. The data and graphs may further comprise average attention span of the test subject 116 while working on a task after completing a second training or phase two 126. The data and graphs may further comprising average attention span of the test subject while working on a task after completing both trainings (i.e., completing phase one 124 and phase two 126. The data and graphs may further comprise the a “retain score” which is the working memory capacity, after completing each session of phase two 126. The data and graphs may further comprise the average retain score (working memory capacity) of the test subject after completing all sessions (for example, all twenty sessions) of phase two 126. The data and graphs may further comprise the performance of the test subject 116 after completing each session of either phase one 124 or phase two 126. The data and graphs may further comprising the average performance of the test subject after completing all sessions of both phase one and phase two.

Based on historical data of each test subject, the system develops a predictive model to calculate an overall “BrainStem” Score. The attributes needed to calculate the overall BrainStem score (Brain Waves, Attention score, Working memory capacity, Performance, Time to complete the task, User Actions (Number of clicks, pressing on the buttons), Emotion Score (which may be identified in a level or score from 1-5). In accordance with one aspect, an exemplary advantage of the present disclosure provides a novel approach to build a Working memory, attention capacity assessment system that help assess and evaluate the child's challenges and reduced the effort excreted by teachers and parents.

The study that led to the development of the brain training system of the present disclosure was guided by two hypotheses. Hypothesis 1: The physical memory brain training matching application disclosed herein will have a better effect on subject engagement and be more efficient and accurate, leading to a better result in training working memory capacity and attention than the non-physical brain training matching application. Hypothesis 2: The intervention will improve learning behavior in the classroom by building awareness among children with poor academic achievement and their understanding of working memory and attention.

The research protocol and consent, and assent forms were approved by the New Mexico State University Institutional Review Board. Before entering the study, written consent was obtained from the parent or legal guardian of each subject, and written assent was obtained from the subject. The study was conducted in a local elementary school. The selection criteria below was utilized to select subjects with the following criteria: (a) poor attention span and high levels of destructibility, (b) poor academic progress in mathematics, (c) demonstrated difficulty in following instructions, (d) demonstrated problems in combining processing with storage, (e) place-keeping difficulty, and (f) short attention span and highly destructible actions.

A total of 18 subjects completed the study: Nine used the AT and WM training devices and nine used the AT and WM training application. Subjects were either eight or nine years old, with no significant age difference between groups. A between-group design was adopted. A blind selection was used for group assignment. Each subject was exposed to only one interface for both training steps.

Subjects were brought to an isolated location in the school library. The setup included a foldable board to isolate the subject to minimize distraction. The subject was seated in front of a laptop that displayed the application and the interface. The subject wore noninvasive BCI, such as device 114.

This research describes the design of the PM-Brain Training Matching Application. This application was highly rated by 85% of the subjects who participated. Subjects were from three local elementary schools and presentations to teachers at these schools about the brain training application also scored highly. Feedback was collected via a questionnaire. A doctor of neurology reviewed the evaluation and feedback that was received from subjects and teachers.

The R Project for Statistical Computing, referred to herein as “R” (https://www.r-project.org) is a statistical computing software and was used to analyze collected data. R is an extremely flexible statistical programming language and environment for statistical computing; it is open source and freely available for all mainstream operating systems.

Attention (AT) data were collected by using the Neurosky brain computer interface; readings ranged from 0 to 100 per second. AT was recorded in the database every second. It was predicted that subjects could increase attention and working memory capacity brainwaves (beta) and suppress drowsiness brainwaves (theta) through training.

Accuracy (n correct) was recorded in the database to represent the number of correct answers per session. Human interaction interface studies have shown that multimodality speeded task completion by 10%, while users made 36% fewer errors. The level of engagement in the domain of the application and control was studied to enhance task performance (time on task and task errors), as well as user rating. They provide improved support for users' preferred interaction style, since 95% to 100% of users prefer multimodal interaction over unimodal interaction.

Efficiency, measured as task completion time (TCT), was recorded and calculated per task. This measure was calculated as the differences between task starting time and task ending time, measured in seconds.

Number of items retained by the subject was recorded for each task completed. This measure was calculated by counting the number of retained items per task and averaging the total per session. For example, if the task was 2, 3, 5 and the subject pressed boxes 2, 3, 5 in the correct order, then the total number of correct items was 3. In the application, visuospatial information can be retained through brainwaves. Subjects' attention feedback was observed based on the screen (WMTA 148) or the LEDs (WMTD 174). Training Session Time (TT) was recorded and calculated as the difference between beginning and ending times of the session. Usability is a key concept in HCl. It is concerned with making systems easy to learn and use. Data on usability (U) were collected via a questionnaire. Many everyday systems and products seem to be designed with little regard for usability, which can lead to frustration on the part of the user. Data on frustration (F) were collected via a questionnaire.

For each of the eleven response variables, two-sample tests for equal mean (or median) were conducted. To address concerns about statistical assumptions, data were first tested for normality using the Shapiro-Wilk test. Here, a significance level of α=0.10 was used (as opposed to α=0.05) to be conservative in terms of more quickly rejecting the normality assumption so as to lean towards more conservative two-sample tests that do not have the normality assumption. If normality was not rejected, Levene's test for equal variances was conducted at a significance level of α=0.10. Depending on the outcome of Levene's test, either a two-sample t-test assuming equal variances was conducted or the Welch two-sample t-test (for unequal variances) was conducted. If normality was rejected, the nonparametric Fligner-Killeen test for equal variances was conducted, as was a bootstrapped Kolmogorov-Smirnov test for testing that the shapes of the underlying distributions are the same. If neither was rejected, then a Wilcoxon Rank Sum test for equal medians was conducted for the two-sample test. If neither a t-test nor a Wilcoxon test had been appropriate, a randomization test would have been conducted, but this was not necessary with any of the eleven response variables. The results of the analyses are summarized in Tables 4, 5, 6.

TABLE 4
Data Analysis for the ATA and ATD using
match-mismatch task.

| | Mean ATD | Mean ATA |
|---|---|---|
| Attention (AT) | 46.67 | 33.78 |
| Accuracy (N_correct) | 0.6856 | 0.3422 |
| Efficiency, measured as a task | 2.34 | 4.136 |
| completion time (TCT) | | |

TABLE 5
Data Analysis for the WMTA and WMTD using the spatial
span task

| | Mean WMTD | Mean WMTA |
|---|---|---|
| Attention (AT) | 49.61 | 36.83 |
| Accuracy (N_correct) | 0.06077 | 0.048 |
| Efficiency, measured as a task | 7.81889 | 12.62778 |
| completion time (TCT) | | |
| Mem. Retained | 4.05556 | 2.43889 |

TABLE 6
Data Analysis summary

| | | | Efficiency | |
|---|---|---|---|---|
| | | | measured | |
| | | | task mem. | Completion |
| | Attention | Accuracy | Retained | time |
| | P-value | p-value | p-value | p-value |
| WMTD and | 0.0427 | 0.1808 | 0.004457 | 0.000716 |
| ATD | | | | |
| WMTA and | 0.002526 | 0.001769 | 0.001234 | |
| ATA | | | | |

The study was concerned with making systems easy to learn and use. A usable system is easy to learn, easy to remember how to use, effective to use, safe to use, and enjoyable to use. Many everyday systems and products seem to be designed with little regard to usability, which can lead frustration, wasted time, and errors. The evaluation questionnaire was designed based on The Fun Sorter and the Again-Again questionnaire (which asks whether the respondent would do the activity again). It asked the subjects to rank items against one or more constructs. This was intended to record their opinions of the technology or activity and to gain a measure of their engagement. 100% of test subjects surface said they would like to participate again when using the physical devices (ATD and WMTD) and the non-physical application based tasks (ATA and WMTA). However, 100% of test subjects indicated that using the physical devices (ATD and WMTD) was helpful in provide attention feedback, whereas only 89% of test subjects indicated that using the non-physical devices (ATA and WMTA) was helpful.

The goal of this project was to design a system interface that would not cause users to become bored, leading to lack of attention, nor so overtasked and stressed as to miss clues or make decision errors. Since the amount of information processing and decision making required in task performance affects the workload experienced by the user, the workload factor was calculated and analyzed carefully.

Subjects participated in pre/post intervention activities. Data analysis showed a significant difference in results between the two tests, indicating a clear increase in performance from pretest to posttest. However, due to the small sample size, these results should be viewed with caution.

FIG. 11 depicts an example of a brain training timeline. The ATD 158 as phase one 124 and WMTD 174 as phase two 126 are shown in the timeline, however it is to be noted that this timeline equally applies to ATA 128 and WMTA 148 when be used as phase one 128 and phase two 126, respectively, to train the brain on how to pay attention and enhance working memory performance. Moreover it should be noted that the physical devices (i.e., ATD, WMTD) are not exclusive to each other. For example, in one instance, the ATD 158 may be utilized for phase one 124, and the WMTA 148 may be utilized for phase two 126. Alternatively, ATA 128 may be utilized for phase one 128 and the WMTD 174 may be utilized for phase two 126.

With continued reference to FIG. 11, the timeline may include an awareness information regarding attention, working memory, and brain awareness for teachers and students as preparation and to change the children's behavior, show generally at 192. The awareness information may be in the form of a teacher generally lecturing to her/his students to let them know about attention and working memory and how the present disclosure believes that their implementation in the manners prescribed herein can lead to an increase in attention and working memory.

The timeline may further include a pre-assessment phase which is shown generally at 194. The pre-assessment may include an assessment given in the form of a test, or quiz, or questionnaire of the test subjects to identify their level of knowledge pertaining to working memory and attention.

FIG. 11 presents that in one aspect, the timeline may be critical to how the system works to improve attention and working memory in a test subject. As discussed above, the first training step (i.e., phase one 124; excluding the pre-assessment steps) of attention based tasks/activities and the second step (i.e., phase two 126) of the working memory tasks can be implemented with this timeline. Furthermore, the whole system is designed to be integrated with the curriculum in a classroom or computer lab in a school.

With continued reference to FIG. 11, the system beings with presentations, such as awareness training 192 and assessments of the students before any attention tasks have been completed (i.e., pre-assessment 194). The pre-assessment 194 is designed to identify terminologies and their definitions when discussing working memory and attention with school-age children (i.e., 5 to 18 years old). Subsequent to the pre-assessment, the system becomes part of the school curriculum, which enables a teacher to lecture about attention and working memory, coincident with the assessments. Typically, the pre-assessment phase is a test or a quiz. In one example, the pre-assessment is set it up was in a school library. However it is possible to have a lab in a classroom that is identified as the “brain training system” or something similar to that effect.

In one example, the system of the present disclosure is account-based; not per-student-based. (i.e., each student has an account and can login to any workstation). For example, about ten students can use the same interface. Students may login as a different user and a control unit or central computer would be a station for the teacher. The teacher would see all of the units she has and then she would turn them on with a control computer so they can use it. And all of these are networked wirelessly to one location.

Feedback 196 monitors the brain and performance activities during phase one 124 and phase two 126. In one example, two weeks of phase one 124 for example of manipulative integrated system attention training, then two weeks of phase two 126 for example of working memory manipulative integrated system. Alternatively, the attention training could be 20 sessions and the working memory training could be 20 session.

A post assessment is completed subsequent to phase one and phase two. The post assessment is shown generally at 198. Then, the system may report and monitoring the cognitive activities and performance could then be evaluated subsequent to training, shown generally at 200. In one example, the system may be used as part of a student's daily activity (for example, at least 20 minutes a day). This timeline is believed to support the position that enhancing working memory may affect the student's math achievement and performance; this will overcome a major STEM (Science, Technology, Engineering and Mathematics) statistics in American society.

In accordance with one aspect, one exemplary non-limiting of the present disclosure is that the interactive system, method, and article of manufacture for training a brain provides a brain training system in schools or outside of school that is available, affordable and interactive (3D), fun, non-invasive, easy to use that provides awareness, assessment, treatment and post assessment to overcome the issue that our nation faces regarding Attention Disorder, low Math achievement, and low working memory performance. The system is overall: unique, interactive, fun to use, and easy to use.

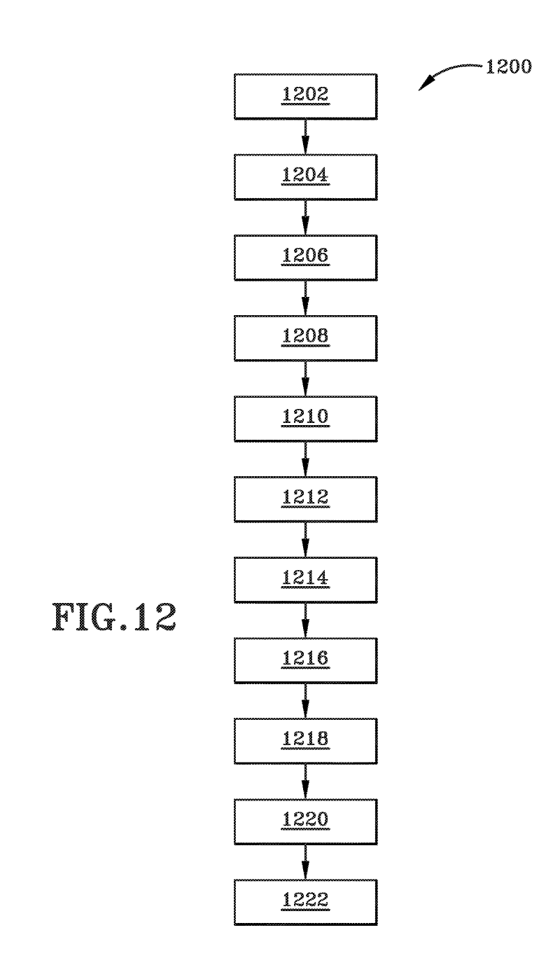

FIG. 12 depicts an exemplarily method of a brain training system in accordance with the present disclosure. A method configured to improve brain functioning is shown generally at 1200. The method 1200 may include equipping a test subject with a neurofeedback monitoring device (such as device 114), shown generally at 1202. The method 1200 may further include providing a first task to be completed, wherein the first task is an attention training/developing technique or task or activity incorporating an image for identification (such as the match/mismatch squares), shown generally at 1204. The method 1200 may further include effectuating the completion of the first task or activity, shown generally at 1206. The method 1200 may further include displaying, in real time, neurofeedback during the completion of the first task to the test subject 116, shown generally at 1208. The method 1200 may further include measuring a completion time of the first task and recording the completion time in a register or database, shown generally at 1210. The method 1200 may further include providing a second task or activity to be completed, wherein the second task is a working memory training developing technique, shown generally at 1212. The method 1200 may further include effectuating the completion of the section task, shown generally at 1214. The method 1200 may further include displaying, in real time, neurofeedback during the completion of the second task to the test subject, shown generally at 1216. The method 1200 may further include measuring a completion time of the second task and recording the completion time in the register, shown generally at 1218. The method 1200 may further include comparing the completion times of the first and second task with predetermined standard completion times, shown generally at 1220. The method 1200 may further include providing a recommendation of attention and working memory of a test subject based on the comparison of the completion times of the first and second task with predetermined standard completion times, wherein if one of the completion times is less than the predetermined standard completion times then indicating one of the following (i) that the test subject is not improving attention span and (ii) that the test subject is not improving working memory, wherein if both the completion times are greater than or equal to the predetermined standard completion times then indicated that the test subject is increasing attention span and working memory, shown generally at 1222.

From the perspective of the test subject 116, some exemplary method steps include: go to the training area; complete pre-test questionnaire for assessment; place the headset in place and make necessary adjustments to ensure proper fit; sign in with your user name and password; read the welcome message and press “Start”; view the objects on the screen, if the two objects match, press the “Match” button, and if they do not match, press the “MisMatch” button, wherein if a green light displays, the answer is correct and if a red light displays, the answer is incorrect; continue to answer for each exercise until you have completed all exercises; log out of the system; remove the headset; complete post-test questionnaire for assessment; review performance and analysis with teacher; and return to your classroom.

From the teacher's perspective, some exemplary method steps include prepare the system, set it up, turn it on, get headset ready to use; retrieve the student (or test subject) from classroom; assist student with pre-test questionnaire for assessment; assist student with installing headset and make necessary adjustments to ensure proper fit; assist student with logging into system; assist student as needed in completing exercises; assist student in logging out of system; assist student with removing headset; assist student with post-test questionnaire for assessment; download or receive student performance and analysis along with the system recommendations and review the same; share student performance and analysis with student and answer any questions; provide positive feedback and coaching; take student back to classroom; file student performance analysis appropriately; and clean system and put it away.

All definitions, as defined and used herein, should be understood to control over dictionary definitions, definitions in documents incorporated by reference, and/or ordinary meanings of the defined terms.

“Logic”, as used herein, includes but is not limited to hardware, firmware, software and/or combinations of each to perform a function(s) or an action(s), and/or to cause a function or action from another logic, method, and/or system. For example, based on a desired application or needs, logic may include a software controlled microprocessor, discrete logic like a processor (e.g., microprocessor), an application specific integrated circuit (ASIC), a programmed logic device, a memory device containing instructions, an electric device having a memory, or the like. Logic may include one or more gates, combinations of gates, or other circuit components. Logic may also be fully embodied as software. Where multiple logics are described, it may be possible to incorporate the multiple logics into one physical logic. Similarly, where a single logic is described, it may be possible to distribute that single logic between multiple physical logics.

Furthermore, the logic(s) presented herein for accomplishing various methods of this system may be directed towards improvements in existing computer-centric or internet-centric technology that may not have previous analog versions. The logic(s) may provide specific functionality directly related to structure that addresses and resolves some problems identified herein. The logic(s) may also provide significantly more advantages to solve these problems by providing an exemplary inventive concept as specific logic structure and concordant functionality of the method and system. Furthermore, the logic(s) may also provide specific computer implemented rules that improve on existing technological processes. The logic(s) provided herein extends beyond merely gathering data, analyzing the information, and displaying the results.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of technology disclosed herein may be implemented using hardware, software, or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer, such as computer 12, may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” or “algorithm” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the preferred embodiment of the disclosure are an example and the disclosure is not limited to the exact details shown or described.

What is claimed:

1. A method configured to improve brain functioning comprising the steps of:
equipping a test subject with a neurofeedback monitoring device;
providing a first task to be completed, wherein the first task is an attention training technique incorporating an image for identification;
effectuating the completion of the first task;
displaying, in real time, neurofeedback during the completion of the first task to the test subject;
measuring a completion time of the first task and recording the completion time in a register;
providing a second task to be completed, wherein the second task is a working memory training technique;
effectuating the completion of the section task;
displaying, in real time, neurofeedback during the completion of the second task to the test subject;
measuring a completion time of the second task and recording the completion time in the register;
comparing the completion times of the first and second task with predetermined standard completion times; and
providing a recommendation of attention and working memory of a test subject based on the comparison of the completion times of the first and second task with predetermined standard completion times, wherein if one of the completion times is less than the predetermined standard completion times then indicating one of the following (i) that the test subject is not improving attention span and (ii) that the test subject is not improving working memory, wherein if both the completion times are greater than or equal to the predetermined standard completion times then indicated that the test subject is increasing attention span and working memory.

2. The method of claim 1, wherein the step of effectuating the completion of the first task is accomplished over a first time frame in a range from one to three weeks.

3. The method of claim 2, wherein the step of effectuating the completion of the second task is accomplished over a second time frame in a range from one to three weeks.

4. The method of claim 1, wherein the step of effectuating the completion of the first task is accomplished over a first time frame in a range from 10 sessions to 30 sessions, wherein in each session is about 20 minutes in length.

5. The method of claim 4, wherein the step of effectuating the completion of the second task is accomplished over a second time frame in a range from 10 sessions to 20 sessions, wherein each session is about 20 minutes in length.

6. The method of claim 5, further comprising:
capturing and recording data from only a portion of the session to normalize results with other test subjects.

7. The method of claim 6, wherein only the results from the first ten minutes of a session is captured.

8. The method of claim 1, wherein the first task to be completed is identifying a match/mismatch between first and second images.

9. The method of claim 8, wherein the first and second images are displayed in a self-contained handheld device.

10. The method of claim 8, wherein the first and second images are displayed on a computer screen and a test subject identifies the match/mismatch via a mouse.

11. The method of claim 1, wherein the working memory training technique comprises the steps of:
illuminating lights in a first sequence;
receiving, via user input, repeated sequential illuminations;
determining whether the repeated sequential illuminations matches the first sequence.

12. The method of claim 11, wherein the first sequence and the repeated sequential illuminations are accomplished entirely in a self-contained handheld device.

13. The method of claim 11, wherein the first sequence and the repeated sequential illuminations are accomplished in a computer application controlled via a mouse or a touch screen.

14. The method of claim 1, wherein the method is implemented in a school curriculum and the test subject is a school-age student.

15. The method of claim 1, wherein effectuating the completion of the first task comprises:
generating, in a first round of testing, a first visual cue and a second visual cue simultaneously, wherein the first completion time begins being measured from the time the first and second visual cues are provided, wherein the first and second visual cues are one of the following: (i) a matched pair, and (ii) a mismatched pair;
receiving a response as to whether first and second cues are a matched pair or mismatched;
determining whether the received response is correct, wherein if the response is correct then increasing difficulty in a second round of testing.

16. A system comprising:
at least one non-transitory computer readable storage medium having instructions encoded thereon, that when executed by one or more processors, perform operations to train attention and working memory in a test subject, the operations comprising:
(i) conduct an attention-based task by presenting first and second images including geometric shapes, wherein the attention-based task requires identification, via user input, as to whether the first and second images match;
(ii) conduct a working memory task by presenting sequenced illuminations, wherein the working memory task requires repetition, via user input, of sequenced illuminations;
a display for displaying at least one of the attention based game/test and the working memory game/test; and
at least one user input device in operative communication with the display.

17. The system of claim 16, wherein the at least one user input device includes a first handheld controller, wherein the display is carried by the first handheld controller; and wherein the first display presents the first and second images during the attention-based task.

18. The system of claim 17, wherein the working memory task is accomplished by the first handheld controller.

19. The system of claim 17, further comprising:
a second handheld controller including at least one light for implementing the illumination sequence of the working memory task.

20. The system of claim 16, further comprising:
at least one attention indicator including a plurality of lights adapted to be illuminated in response to neurofeedback information generated by the test subject;
wherein the plurality of lights are illuminated in real-time so as to allow the test subject to visualize their attention during at least one of (i) the attention-based task and (ii) the working memory-based task; and
wherein the lights are positioned below an object shaped in the form of a human brain.
